(19) United States
(12) Patent Application Publication
(10) Pub. No.: US 2008/0299131 A1
(43) Pub. Date: Dec. 4, 2008

(54) Anti-Viral Compositions

(75) Inventors: Geoffrey Lilley Smith, Nr Oxford (GB); Gemma Chevonne Carter, Warwick (GB); Mansun Law, La Jolla, CA (US); Michael Stanley Hollinshead, Abingdon (GB)

Correspondence Address: Leon R Yankwich;Yankwich & Associates, 201 Broadway, Cambridge, MA, 02139, US

(73) Assignee: IMPERIAL INNOVATIONS LIMITED, London (GB)

(21) Appl. No.: 11/631,088

(22) PCT Filed: Jun. 28, 2005
(86) PCT No.: PCT/GB2005/002527

§ 371 Date: Apr. 23, 2007

(30) Foreign Application Priority Data
Jun. 28, 2004 (GB) 0414441.6

(51) Int. Cl. A61K 39/395; C12N 7/06; A61P 31/12

(52) U.S. Cl. 424/159.1; 435/238

(57) ABSTRACT

The invention provides a composition comprising for simultaneous, sequential or separate administration a) a polyanion; and b) an antibody reactive against an antigen on the surface of an intracellular form of a virus, which virus has an extracellular form that is surrounded by one lipid membrane more than the intracellular form. The present inventors have found that the compositions according to the invention comprising an antibody and a polyanion can neutralize virus infectivity more efficiently than other compositions reported hitherto.

The invention relates to compositions for the treatment of viruses and virus infections, and the use of such compositions. The invention finds particular application with viruses which exist in an intracellular form and an extracellular form, the extracellular form being surrounded by one membrane more than the intracellular form, for example poxviruses.

### INTRODUCTION

Enveloped viruses which exist in an intracellular form and an extracellular form that is surrounded by one membrane more than the intracellular form, include, for example, poxviruses and African swine fever virus. The additional membrane helps protect the intracellular form from antibody, complement and other anti-viral compounds.

Poxviruses are a family of large viruses that replicate in the cell cytoplasm (Moss, 2001). The Poxviridae are subdivided into viruses that infect insects, Entomopoxviruses, and chordates, Chordopoxviruses. The Chordopoxvirinae is divided into 8 genera of which the Orthopoxvirus genus has been the most important for humans (Fenner et al. 1989). This genus contains Variola virus, the cause of the disease smallpox, Vaccinia virus (VACV) the vaccine used to prevent smallpox, Monkeypox virus, the cause of monkeypox, Cowpox virus, Camelpox virus and Ectromelia virus, the cause of mousepox. Properties of these viruses include a large and complex virus particle (250×350 nm), a double stranded DNA genome of roughly 200,000 base pairs that encodes about 200 genes, replication in the cytoplasm, virus-encoded enzymes for the processes of transcription and DNA replication, and many virulence factors that are non-essential for virus replication in cell culture, but which affect the outcome of infection in vivo (Moss, 2001). Members of the genus are morphologically indistinguishable and antigenically cross-reactive such that prior infection with any member of the genus protects against subsequent infection by any other member of the genus (Fenner et al., 1989). Viruses from other chordopoxvirus genera that infect man are Orf virus (a parapoxvirus), Molluscum contagiosum virus (a molluscipox virus) and Tanapox (a Yatapoxvirus). Other chordopoxviruses infect animals and may cause economically important diseases. For instance, capripoxviruses (goatpox virus, sheepox virus and lumpy skin disease virus) infect goats, sheep and cattle, suipoxvirus (swinepox virus) infects swine, and leporipoxviruses (myxoma virus and Shope fibroma virus) infect rabbits. Other parapoxvirus infect cattle and reindeer. The present invention finds application with all these viruses.

The replication of orthopoxviruses produces two forms of infectious virus called intracellular mature virus (IMV) and extracellular enveloped virus (EEV). IMV particles represent the great majority of infectious progeny and when freeze-dried resist physical forces and elevated temperatures that kill many other viruses. IMV particles constitute the infectious virus present in smallpox vaccines and are believed to transmit infection between hosts. IMV are released from cells very late after infection due to cell lysis and are insufficient for efficient cell-to-cell spread of infection (Smith et al., 2003).

EEV particles are much less abundant than IMV (less than 1% of infectivity with some strains of VACV) and represent IMV particles that are wrapped in an additional lipid membrane that contains several virus-encoded proteins that are absent from IMV. Although only a minor component of total infectivity, EEV particles are important for two reasons. First, they are responsible for virus dissemination within a host and are more resistant to neutralization by antibody or complement than IMV (Vanderplasschen et al., 1998b). Second, for immunological protection against poxviruses it is necessary to have immunity against the antigens present in the EEV outer envelope and which are absent form MV (Boulter & Appleyard, 1973). It was for this reason that candidate vaccines for smallpox that were based on only inactivated IMV were ineffective. Antibodies against IMV antigens are less effective at preventing poxvirus infection than antibodies against EEV. The outer EEV envelope contains several proteins that are absent form IMV (Smith et al., 2002). These are encoded by genes F13L, A33R, A34R, A56R and B5R. In addition there are two proteins, encoded by genes F12L and A36R, that are present on intracellular enveloped virus (IEV) but absent from IMV and EEV. These proteins have been termed transport proteins because they function to transport the infectious virus particles to the cell surface and out of the cell by utilizing microtubules and actin components of the cell (Smith et al., 2002). The functions of the EEV proteins have been studied by the construction of virus mutants lacking these genes. In the absence of F13L or B5R virus morphogenesis arrests before the wrapping of IMV particles to form intracellular enveloped virus (IEV). Without F12L, IEV particles are formed but are not transported to the cell surface. In contrast, mutants lacking A33R, A34R and A36R move to the cell surface but fail to induce virus-tipped actin tails that are important for cell-to-cell spread. Lastly, loss of the A56R gene does not affect these processes.

The only EEV protein that has been identified as a target for neutralizing antibody is B5R (Galmiche et al., 1999; Law & Smith, 2001), although antibody to A33R can induce some protection against disease (Galmiche et al., 1999; Hooper et al., 2000). However no neutralizing monoclonal antibody (mAb) against EEV has been found (Law & Smith, 2001) and there is no efficient means to neutralize EEV by antibody. EEV mediates long-range virus spread both in vitro and in vivo.

How EEV or IMV particles bind to cells is largely unknown, although the receptors on cells for these virions are different (Vanderplasschen & Smith, 1997). This is consistent with the presence of different proteins on the surface of these virions. It was reported that glucosarninoglycans (GAGs) were cell receptors for IMV because some of these compounds could reduce binding of IMV particles to cells, and different IMV proteins were reported to bind to some of these compounds, see below. However, the effect of these compounds on EEV binding was not investigated.

African swine fever virus (ASFV) is a large DNA virus that has some similarities with poxviruses and iridoviruses. It shares a similar genome structure, site of replication and transcriptional enzymes with poxviruses, but has an icosahedral capsid reminiscent of Iridoviruses. It is classified as the sole member of the Asfravirus family. ASFV replicates in the cytoplasm and produces an intracellular virion that is infectious and contains at least one membrane derived from the endoplasmic reticulum. This virus can either be released from cells when they lyse, or it can bud through the plasma membrane before cell death and acquire an additional lipid envelope that is absent from the intracellular form of virus. This extracellular form of virus is poorly characterized but is thought to contain proteins in its outer envelope that are absent from the intracellular form. ASFV has proved difficult to neutralize with antibody derived from animals that have recovered from infection with ASFV (Zsak et al., 1993; Gomez-Puertas et al., 1996; Gomez-Puertas & Escribano, 1997). ASFV that has been passaged in cell culture becomes more resistant to neutralization by antibody (Gomez-Puertas et al., 1997).

### SUMMARY OF THE INVENTION

The invention provides a composition comprising for simultaneous, sequential or separate administration

a) a polyanion; and
b) an antibody reactive against an antigen on the surface of an intracellular form of a virus, which virus has an extracellular form that is surrounded by one lipid membrane more than the intracellular form.

The present inventors have found that the compositions according to the invention comprising an antibody and a polyanion can neutralize virus infectivity more efficiently than other compositions reported hitherto.

The invention also provides a composition for the treatment of a subject infected with a virus, which virus has an extracellular form and an intracellular form, the extracellular form being surrounded by one lipid membrane more than the intracellular form whereby the subject is a subject that has previously raised an immune response against an antigen on the surface of an intracellular form of the virus.

The present inventors have found that such compositions can enable the immune response of a patient in response to infection to be more effective in protecting against or dealing with disease.

The invention also relates to methods and uses which exploit the beneficial properties of compositions of the invention as described in further detail below.

### BRIEF DESCRIPTION OF THE FIGURES

FIG. 1 shows the synergistic effect of human antisera and polyanions on the neutralisation of VACV.

FIG. 2 shows inhibition of EEV infectivity with IMV neutralising mAb in the presence of polyanions.

FIG. 3 shows the synergistic effects of various polyanions and of IMV neutralising mAb (mAb 2D5) on the neutralization of EEV.

FIG. 4 shows the results of anti-comet assays using polyanions and of mAb 2D5

FIG. 5 shows the effects of the presence of HP on the binding and entry of EEV

FIG. 6 shows the effect of polyanion on virus production.

FIG. 7 shows the effect of polyanions on the formation of VACV-induced actin tails.

FIG. 8 shows electron micrographs of EEVs incubated with and without HP.

FIG. 9 shows electron micrographs of RK₁₃ cells infected with VACV in the presence and absence of HP

FIG. 10 shows the effects of anti-VACV antibody and HP on VACV infection in vivo.

FIG. 11 shows the titre of virus present in the lungs, brains and spleens of VACV-infected mice treated with PA and rabbit anti-IMV antibody (Rb anti-IMV Ab).

FIG. 12 shows the effects of anti-VACV antibody and HP on VACV infection in vivo.

FIG. 13 shows a hypothetical mechanism of how the EEV membrane is ruptured.

FIG. 14 shows the inhibition by polyanions of EEV made by VACV mutants lacking individual EEV proteins.

FIG. 15 shows the inhibition by polyanions of EEV made by VACV mutants with alterations in the B5R protein.

### DETAILED DESCRIPTION

The invention is based on the observation that polyanionic compounds can influence the interaction of elements of the immune system, particularly antibodies, with antigens on the surface of an intracellular form of a virus, which virus has an extracellular form that is surrounded by one lipid membrane more than the intracellular form. In the case of VACV, which exists in the forms of extracellular enveloped virus (EEV) and intracellular mature virus (IMV) particles, the polyanionic compound is able to disrupt the outer envelope of EEV particles so that antibodies to IMV antigens are able to gain access to the IMV surface and neutralize virus infectivity.

Preferably, the virus is a double stranded DNA virus.

Amongst viruses which have an extracellular form that is surrounded by at least one lipid membrane more than the intracellular form, the invention finds particular application with the chordopoxviruses. As mentioned above, the chordopoxviruses include the orthopoxviruses, the parapoxviruses (for example Orf virus), the molluscipox viruses (for example Molluscum contagiosum virus), Yatapoxvirus (for example Tanapox virus), which infect man and also capripoxviruses (for example goatpox virus, sheepox virus and lumpy skin disease virus) infect goats, sheep and cattle, suipoxvirus (for example swinepox virus) which infects swine, and leporipoxviruses (for example myxoma virus and Shope fibroma virus) which infect rabbits. Other parapoxvirus infect cattle and reindeer. The present invention finds application with all of those viruses. It finds particular application with the viruses that infect humans.

Of the chordopoxviruses, the orthopoxviruses are particularly suitable. The orthopoxvirus genus contains, amongst others, Variola virus, the cause of the disease smallpox, VACV the vaccine used to prevent smallpox, Monkeypox virus, the cause of monkeypox, Cowpox virus, Camelpox virus and Ectromelia virus, the cause of mousepox. In the examples herein, the invention is illustrated with work with VACV.

The invention finds particular application with the orthopoxviruses that infect humans. In addition, the invention also finds application in the treatment of infections of non-human mammals by orthopoxviruses, including cowpox, monkeypox virus, camelpox virus and other chordopoxviruses such as orf virus and molluscum contagiosum and Yatapoxvirus. For example, the invention may be used in the treatment of mice against ectromelia, camels against camelpox, a wide range of mammals, for example rodents, cats, cows, large felines or elephants against cowpox, or rodents or monkeys against monkeypox.

As used herein the term “polyanion” is understood to refer to a polymer molecule which carries a plurality of negative charges when in aqueous solution at or around physiological pH. Such polymers typically have a molecular weight Mr of at least 400, preferably at least 800. Typically, polymers for use in the invention have a molecular weight Mr of less than 5,000,000, preferably less than 1,000,000, for example less than 750,000. The polyanionic polymer thus preferably has an Mr of from 400 to 1,000,000, more preferably from 800 to 750,000, more preferably from 1500 to 500,000. The exact molecular weight that is most appropriate depends on the nature of the polyanion. Optimisation of the molecular weight of a particular polyanion for a particular application lies within the competence of the person skilled in the art. In the case of heparin, the polyanionic polymer may, for example, have an Mr of from 1,000 to 50,000, preferably from 2,000 to 20,000, for example from 4,000 to 6,000 or from 10,000 to 20,000, for example 15,000. In the case of dextran sulphate, the polyanionic polymer may, for example, have an Mr of from 2,000 to 750,000, for example from 4,000 to 6,000 or from 250,000 to 750,000, for example 5,000 or 500,000.

A single polyanion may be used in the invention. Alternatively, it may be preferable to use a mixture of two or more different polyanions.

The effectiveness of the polyanions (PAs) is dependent upon their size (molecular weight), charge density and total charge. Polyanionic polymers with a greater size and charge were found to be more effective in the context of this invention. On the other hand high Mr polyanionic polymers may have adverse effects on the subject in the case of some PAs, so a balance must typically be found. In experiments described herein, it was found that heparin (HP) high molecular weight heparin (“HP-HMW”) with Mr=15,000 was more effective than heparin with lower molecular weight (“HP”) with molecular weight 4000-6000. Similarly, in experiments using dextran sulphate (DS), high molecular weight DS (“DS-HMW”) derived from dextran with Mr=500,000 was found to be more effective than DS with lower molecular weight (“DS” derived from dextran with Mr=5,000).

Polyanions include natural and synthetic polyanionic polymers such as polysaccharides and naphthalene polymers, for example sulphated polysaccharides and naphthalene polymers and their derivatives. Sulphated polysaccharides include dextran sulphate, cellulose sulphate, heparin or heparan sulphate, dermatan sulphate, chondroitin sulphate, pentosan sulphate, fucoidin, mannan sulphate, carrageenan, dextrin sulphate, curdlan sulphate and chitin sulphate and their derivatives. The polysaccharides may be homo- or heteropolysaccharides The monomeric units may be, for example, aldo-, deoxyaldo-, keto- or deoxyketopentoses including but not limited to arabinose, ribose, deoxyribose, galactose, fructose, sorbose, rhamnose and fucose, joined by either alpha- or beta-linkages. The polymer can be linear or branched, with free hydroxyl groups of the monomeric units maximally or partially sulphated.

A further example of a suitable polyanion is the polynaphthylene compound sold under the tradename “PRO2000”, developed by Procept and Indevus Pharmaceuticals Inc, of 99 Hayden Avenue, Suite 200, Lexington, Mass. 02421, USA (previously Interneuron, Inc.).

The polyanionic polymers for use in the invention may be in the form of a salt or a solvate or other pharmaceutically acceptable physiologically functional derivative. Salts and solvates which are suitable for use in medicine are those wherein a counterion or associated solvent is pharmaceutically acceptable. By the term “physiologically functional derivative” is meant a chemical derivative of a compound of formula (I) having the same physiological function as the free compound of formula (I), for example, by being convertible in the body thereto. According to the present invention, examples of physiological functional derivatives include esters, amides, and carbamates; preferably esters and amides.

Pharmaceutically acceptable base salts include ammonium salts, alkali metal salts, for example those of potassium and sodium, alkaline earth metal salts, for example those of calcium and magnesium, and salts with organic bases, for example dicyclohexylamine and N-methyl-D-glucomine.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”.

A compound which, upon administration to the recipient, is capable of providing (directly or indirectly) a polyanion as described above or an active metabolite or residue thereof, is known as a “prodrug”. A prodrug may, for example, be converted within the body, e.g. by hydrolysis in the blood, into its active form that has medical effects. Pharmaceutical acceptable prodrugs are described in T. Higuchi and V. Stella, Prodrugs as Novel Delivery Systems, Vol. 14 of the A. C. S. Symposium Series; and in Edward B. Roche, ed., Bioreversible Carriers in Drug Design, American Pharmaceutical Association and Pergamon Press, 1987, both of which are incorporated herein by reference.

In a first aspect the invention provides a composition comprising an antibody reactive against an antigen on the surface of an intracellular form of a virus, which virus has an extracellular form that is surrounded by one lipid membrane more than the intracellular form. In compositions for the treatment of orthopoxviruses, the antibody is reactive against neutralizing epitopes/antigens on the surface of IMV. Known antigens are A17L, A27L, H3L, L1R and D8L of VACV and their orthologues in other poxviruses. Known antibodies are, for example human vaccinia-immune globulin (VIG), recombinant IMV neutralizing human scFv, Fab or mAb (Schmaljohn et al., 1999; Tikunova et al., 2001), mouse mAb 2D5 (Ichihashi & Oie, 1996) against the L1R protein, and mouse mAb C3 against the A27L protein (Rodriguez et al., 1985). The production and characterization of mAbs to IMV proteins has been described by Rodriguez et al, 1985. When used alone, these antibodies are ineffective against EEV because they cannot gain access to IMV. In the compositions of the invention, the PA compound enables the antibodies to IMV antigens to gain access to their targets.

The antibodies may be monoclonal or polyclonal antibodies. For use in the treatment of human subjects, the antibodies may be humanised.

The invention further provides compositions of the invention for use as a medicament. The medicaments may be used as therapeutic agents to prevent or ameliorate disease caused by viruses, for example chordopoxviruses in man and animals.

Compositions of the invention comprising an antibody reactive against an antigen on the surface of an intracellular form of a virus, which virus has an extracellular form that is surrounded by one lipid membrane more than the intracellular form, have use in the treatment of infection caused by that virus. For example, they are useful in the treatment of infection, for example of mammals, such as humans, by orthopoxviruses, including variola virus, monkeypox virus, cowpox virus and VACV. They are particularly useful for the treatment of infection of humans by variola virus, monkeypox virus, VACV and cowpox virus. The invention also finds application in the treatment of other chordopoxviruses that infect man or animals as described above. It is not essential that the virus to be treated is the same virus against which the antibody in the composition of the invention was raised. Generally, however, the antibodies should be antibodies raised against a virus of the same genus as the virus to be treated. It is, preferred that the virus to be treated is the same virus against which the antibody in the composition of the invention was raised.

The compositions of the invention comprising an antibody reactive against an antigen on the surface of an intracellular form of a virus, which virus has an extracellular form that is surrounded by one lipid membrane more than the intracellular form also have use in the treatment of infection caused by other viruses that are surrounded by a double membrane, such as African swine fever virus, the agent of an economically important disease of domestic pigs.

The invention also provides the use of a composition of the invention for the manufacture of a medicament for the treatment of an infection of a mammal by a virus, which virus has an extracellular form that is surrounded by one lipid membrane more than the intracellular form.

The invention also provides a method of treating a subject infected with a virus, which virus has an extracellular form that is surrounded by one lipid membrane more than the intracellular form, comprising administering to the subject an effective amount of such a composition.

The components of the compositions of the invention may be administered simultaneously, sequentially or separately. Compositions for simultaneous administration may comprise the two components in pre-mixed form or the components may be in separate administration units with instructions for simultaneous administration. The antibody component may be administered by the same route as the polyanion, or the two components may be administered by different routes.

With regard to treatment of humans exposed to Variola virus, the cause of smallpox, it is known that vaccination within the first 4 days after exposure provides some benefit, but the benefit is greater the sooner the vaccine is given after exposure, and after day 4 it is too late.

Mice infected intranasally with VACV strain Western Reserve (WR) develop disease and this may be prevented by passive immunization with antibodies to EEV, but antibodies to IMV are less beneficial. However, the present inventors have now found that, if mice are immunized passively with anti-IMV antibodies, and infected intranasally and then treated with polyanions, the disease is prevented. Importantly, polyanion therapy several days after infection still confers protection against disease.

Accordingly, the polyanion and the antibody may be administered simultaneously, for example as a mixture, or sequentially. The compositions of the invention are also effective if the two components are administered some time apart, for example from one hour to 2 months apart, such as from 12 hours to 1 month apart, for example from 1 to 20, 1 to 17, 1 to 9 or 1 to 14 days apart.

In a mouse model, animals that had been injected with anti-IMV antibody and then infected with VACV and then given PAs 2-days later were protected from disease, despite the fact that disease was apparent in the absence of treatment only 2 days later. It may be possible to confer benefit by administration of PAs and antibodies even later. Smallpox takes between 9 and 17 days to develop after exposure (Fenner, 1988), and so it is expected to be possible to deliver benefit to those exposed to variola virus by administering PAs and antibody later than would be possible by vaccination. This is because PAs in the presence of anti-IMV antibody act immediately and do not require the vaccine to induce an immune response that would take days to develop.

The conventional smallpox vaccines (e.g. Lister or New York City Board of Health) are known to cause rare serious complications such as eczema vaccinatum, progressive vaccinia or neurological complications (Fenner, 1988) and traditionally vaccinia immune globulin (VIG) was given to patients suffering such severe reactions to vaccination to treat these conditions. The therapeutic value of VIG is incomplete and there are clinical cases in which VIG was unable to control virus progression (Bray & Wright, 2003). VIG is a pool of hyperimmune human anti-VACV antibody. The neutralizing activity of VIG is mostly determined and normalized against IMV rather than EEV (Anderson & Skegg, 1970) despite the fact that antibody against EEV is more potent (Boulter & Appleyard, 1973; Galmiche et al., 1999; Law & Smith, 2001; Smith et al., 2002; Earl et al., 2004). The VIG used in those cases might have a very high IMV-neutralizing titre but the anti-EEV activity is often uncertain. The finding that PAs disrupt the EEV membrane provides an excellent opportunity to provide improved treatments and vaccines for patients suffering severe reaction to vaccination. Polyanionic polymers together with IMV-neutralizing human mAbs or a vaccine composition may be a beneficial alternative to VIG.

When administered at the site of virus replication in a poxvirus-pneumonia model, PAs offered a significant protection to the host and administration of the PAs could be delayed until after infection, shortly before disease became evident. PAs act synergistically with antibody to inhibit poxvirus infection or disease.

The invention also provides a kit comprising in separate compartments

a) a polyanion; and
b) an antibody reactive against an antigen on the surface of an intracellular form of a virus, which virus has an extracellular form that is surrounded by one lipid membrane more than the intracellular form.

The components of the kit are preferably as described above in relation to the compositions of the invention.

In a second aspect, the invention provides the use of a polyanion for the manufacture of a medicament for the treatment of a subject infected with a virus, which virus has an extracellular form and an intracellular form, the extracellular form being surrounded by one lipid membrane more than the intracellular form whereby the subject is a subject that possesses antibodies against an antigen on the surface of an intracellular form of the virus.

The antibodies that the subject possesses may be present in the subject because they were previously administered into the subject. Alternatively, they may be present by virtue of an immune response by the subject, for example in response to infection by the virus or a related virus, or in response to vaccination against the virus or a related virus. The presence of appropriate antibodies in the subject may be assessed by conventional means, for example by way of an ELISA assay.

For the case in which the antibodies are present in the subject by virtue of an earlier vaccination, any suitable known vaccine against the disease in question may have been used. In the case of a vaccine against a virus of the poxvirus family, the vaccine may be an orthopoxvirus or a derivative thereof, preferably a VACV, a cowpox virus, a camelpox virus or an ectromelia virus or a derivative of any of those viruses. Most preferably, the vaccine is a VACV. A VACV may be a VACV strain selected from the group consisting of Lister, Copenhagen, Wyeth, New York City Board of Health, NYVAC, Praha virus, DRYVAX Wyeth-derived virus, LIVP, IHD-J, IHD-W, Tian Tan, Tashkent, King Institute, Patwadanger, EM-63, Evans, Bern, LC16m0 or MVA. Preferably, a VACV strain is selected from the group consisting of MVA, Lister, New York City Board of Health, Copenhagen or Wyeth. It is not essential that the virus to be treated is the same virus as used in the vaccination. Generally, however, the vaccine should be against a virus of the same genus as the virus to be treated.

The invention thus further provides a composition comprising a polyanion for the treatment of a subject infected with a virus, which virus has an extracellular form and an intracellular form, the extracellular form being surrounded by one lipid membrane more than the intracellular form whereby the subject is a subject that possesses antibodies against an antigen on the surface of an intracellular form of the virus. The invention also provides a method of treating a subject infected with a virus, which virus has an extracellular form and an intracellular form, the extracellular form being surrounded by one lipid membrane more than the intracellular form, whereby the subject is a subject that possesses antibodies against an antigen on the surface of an intracellular form of the virus, comprising the step of administering to the subject in need thereof a composition comprising a polyanion.

The invention also provides a method of treating a subject comprising the steps of
a) administering to the subject
   (i) a vaccine against a virus, which virus has an extracellular form and an intracellular form, the extracellular form being surrounded by one lipid membrane more than the intracellular form; or
   (ii) an antibody against a virus, which virus has an extracellular form and an intracellular form, the extracellular form being surrounded by one lipid membrane more than the intracellular form; and
b) administering to the subject a polyanion.

The polyanion is preferably administered after antibodies are present in the subject, for example after an immune response has been raised in response to vaccination. The vaccine or antibody may be administered before or after infection with the virus, for example before infection with the virus. Administration of polyanion makes antibodies (either administered antibodies or antibodies raised in the subject) against the virus more effective.

The conventional smallpox vaccines (e.g. Lister or New York City Board of Health) are known to cause rare serious complications such as eczema vaccinatum, progressive vaccinia or neurological complications (Fenner, 1988) and traditionally vaccinia immune globulin (VIG) was given to patients suffering such severe reactions to vaccination to treat these conditions. The therapeutic value of VIG is incomplete and there are clinical cases in which VIG was unable to control virus progression (Bray & Wright, 2003). VIG is a pool of hyperimmune human anti-VACV antibody. The neutralizing activity of VIG is mostly determined and normalized against IMV rather than EEV (Anderson & Skegg, 1970) despite the fact that antibody against EEV is more potent (Boulter & Appleyard, 1973; Galminche et al., 1999; Law & Smith, 2001; Smith et al., 2002; Earl et al., 2004). The VIG used in those cases might have a very high IMV-neutralizing titre but the anti-EEV activity is often uncertain. The finding that PAs disrupt the EEV membrane provides an excellent opportunity to provide improved treatments and vaccines for patients suffering severe reaction to vaccination. Polyanions together with IMV-neutralizing human mAbs or a vaccine composition may be a beneficial alternative to VIG.

When administered at the site of virus replication in a poxvirus-pneumonia model, PAs offered a significant protection to the host and administration of the PAs could be delayed until after infection, shortly before disease became evident. PAs act synergistically with antibody to inhibit poxvirus infection or disease.

For other enveloped viruses including many that infect the respiratory system primarily (see introduction), it has been found that they can be inhibited efficiently by PAs per se. Owing to the undesirable properties of these compounds when administered intravenously, intraperitoneally and subcutaneously, their use in treating respiratory viral diseases has not been investigated. The present inventors have demonstrated that direct administration of PAs to the respiratory route can be used to treat pneumonia caused by a virus, in this case VACV. Accordingly, the invention provides a composition comprising a polyanion for use in the treatment of a disease of the respiratory tract. Such compositions find particular application in the treatment of diseases of the upper respiratory tract.

There are many clinical trials using VACV or genetically engineered variants thereof. Safety testing of these vaccines batches requires a demonstration that other infectious agents are absent. This has proved very hard to do because it is technically very difficult to neutralize all the VACV infectivity due to the inherent resistance of EEV and CEV to neutralization. When the sample being tested is incubated with suitable cells, very often these cells are destroyed by the residual VACV infectivity and so other agents are undetectable. The compositions of the present invention provide a way to neutralize VACV infectivity more easily and completely so that other infectious agents can be detected. The invention thus further provides a method of neutralizing in vitro the infectivity of a virus, which virus has an extracellular form that is surrounded by one lipid membrane more than the intracellular form comprising the step of combining a test sample with a composition comprising

a) at least one polyanion; and
b) an antibody reactive against an antigen on the surface of an intracellular form of a virus, which virus has an extracellular form that is surrounded by one lipid membrane more than the intracellular form.

The composition is particularly useful for the neutralising the infectivity of VACV. This is useful because EEV is notoriously hard to neutralize and it has proved very difficult to remove all VACV infectivity during safety testing of vaccines batches containing VACV. This is necessary to demonstrate that the vaccine is free of other infectious agents.

### Mechanistic Considerations

A possible mechanism for the beneficial effects of the compositions of the invention is that the polyanionic compound is able to disrupt the outer lipid membrane of the extracellular enveloped virus (EEV) particle thus allowing antibodies reactive against an antigen on the surface of the intracellular form of the virus (IMV) to gain access to the surface of the intracellular form of the virus and thus neutralize virus infectivity. In addition, the compositions of the invention inhibit the formation of actin tails from the surface of infected cells and thereby reduce dissemination of virus to surrounding cells. The utility of these compounds has been demonstrated in both cell culture and in vivo.

Polyanions (PAs) such as dextran sulphate (DS) and heparin (HP) are known to have an inhibitory effect on many families of enveloped viruses including human immunodeficiency virus (HIV), herpes simplex virus (HSV), influenza virus, respiratory syncytial virus, measles virus and parainfluenza viruses (Lüscher-Mattli, 2000; De Clercq, 2001). The antiviral activities of these compounds had been studied intensively, demonstrating that virus infections are blocked at the stages of virus attachment and membrane fusion (Mitsuya et al., 1988; Lüischer-Mattli et al., 1993; Gordon et al., 1995). The broad and potent anti-viral activity of PAs in vitro had attracted intensive research but the failure in clinical trials had prevented further development of PAs as antiviral therapeutics. Recent advances in the design and modification of PAs had reduced some of the undesirable properties and compounds like dextrin sulphate and PRO2000 are now in clinical trials as topical and therapeutic antiviral agents to HIV infection.

The mechanism of action of polyanionic polymers with anti-viral activity against HIV, HSV and other viruses has been based on their observed ability to inhibit virion binding to the target cells. The disruption of a viral membrane by a polyanionic polymer has not been reported in enveloped virus families.

Previously DS and HP were found to have a weak inhibitory effect on poxviruses including VACV (Witvrouw et al., 1994; Chung et al. 1998). Those studies also suggested that poxviruses may utilize cell surface GAGs for the initial virus binding in a manner resembling HSV. However, the inhibition was only partial and inefficient, and the virus used in the studies was IMV that is not responsible for virus spread during an infection. Despite the fact that EEV is essential for efficient virus spread (Boulter & Appleyard, 1973; Smith et al., 2003), hitherto there is no study of whether or not PAs affect EEV.

EEV has an additional host-derived lipid membrane to protect the internal highly immunogenic IMV from antibody and complement. Several EEV-specific proteins were identified and passive transfer of antibodies against B5R or A33R protected mice from virus challenge (Galmiche et al., 1999; Hooper et al., 2000). However no neutralizing mAb against EEV has been found (Law & Smith, 2001) and there is no effective means to neutralize EEV by antibody. EEV mediates long-range virus spread both in vitro and in vivo and the structurally indistinguishable virus form, cell-associated virus (CEV), induces actin tail formation at the cell surface to drive the virion into neighboring cells for efficient cell-to-cell spread. To achieve effective antiviral activity, it is necessary to target EEV and CEV during the course of virus infection (Boulter & Appleyard, 1973; Earl et al. 2004).

The binding and entry mechanism of poxviruses are unclear. IMV has been suggested to enter cells by direct fusion of virus and plasma membrane to release the virus core into cytosol (Armstrong et al., 1973) but others proposed that the IMV membrane(s) uncoated at the cell surface and the core was somehow injected into cytosol by an unknown mechanism (Sodeik & Krijnse-Locker, 2002). In either model EEV has to shed one more membrane than IMV for the virus core to gain access to cells. EEV particles were proposed to enter cells via endosomes in which the low pH environment damaged the fragile EEV membrane and released an MV within to fuse with the endosomal membrane (Ichihashi, 1996; Vanderplasschen et al., 1998a). In another study, EEV was reported to enter cells directly from cell surface by an unknown mechanism (Krijnse-Locker et al., 2000).

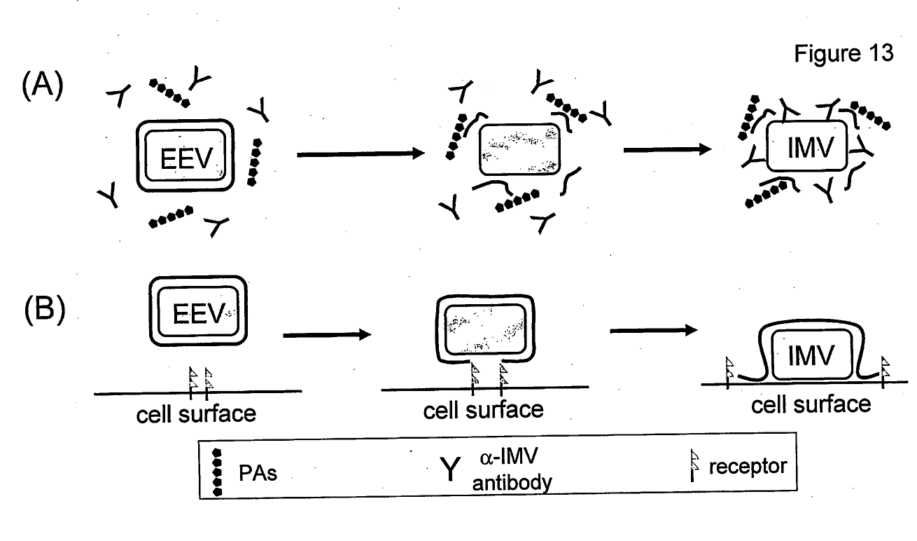

In both models shedding the EEV membrane is a critical step to release IMV for entry. PAs are more effective than reduced pH at destroying the outer membrane and do not affect virus infectivity upon incubation. The activity is specific to the EEV membrane because PAs do not affect IMV and plasma membranes. It is possible that PAs share a similar mechanism to cell ligands or receptors that interact with specific EEV proteins responsible for the shedding of EEV membrane (FIG. 13 shows a cartoon illustrating how PAs break the EEV membrane and how the EEV membrane ruptures upon cell contact to release IMV). Since low pH is not the most efficient factor for shedding of EEV membrane, endosomes may not be the actual entry pathway for EEV. A possibility would be that EEV binds to cell surface GAGs such as heparan sulphate or chondroitin sulphate and release IMV at cell surface for penetration.

As described above, the invention provides pharmaceutical compositions. The amount of active ingredient which is required to achieve a therapeutic effect in a composition will, of course, vary with the particular compound, the route of administration, the subject under treatment, and the severity of particular infection being treated. The compositions of the invention may be administered orally or via injection at a dose for each component of from 0.1 to 1500 mg/kg per day, preferably 0.1 to 500 mg/kg per day. The dose range for adult humans is generally from 5 mg to 35 g per day and preferably 5 mg to 2 g per day. Tablets or other forms of presentation provided in discrete units may conveniently contain an amount of compound of the invention which is effective at such dosage or as a multiple of the same, for example units containing 5 mg to 500 mg, usually around 10 mg to 200 mg.

While it is possible for the active ingredient to be administered alone, it is preferable for it to be present in a pharmaceutical formulation. Accordingly, the invention provides a pharmaceutical formulation comprising the active ingredient and a pharmaceutically acceptable excipient. The pharmaceutical formulations according to the invention include those suitable for oral, parenteral (including subcutaneous, intradermal, intramuscular, intravenous, and intraarticular), inhalation (including fine particle dusts or mists which may be generated by means of various types of metered does pressurized aerosols), nebulizers or insufflators, rectal and topical (including dermal, buccal, sublingual, and intraocular) administration, although the most suitable route may depend upon, for example, the condition and disorder of the recipient.

The formulations may conveniently be presented in unit dosage form and may be prepared by any of the methods well known in the art of pharmacy.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water for injection, immediately prior to use. Extemporaneous injection solutions and suspensions may be prepared from sterile powders, granules and tablets of the kind previously described. Exemplary compositions for nasal aerosol or inhalation administration include solutions in saline, which can contain, for example, benzyl alcohol or other suitable preservatives, absorption promoters to enhance bioavailability, and/or other solubilizing or dispersing agents such as those known in the art.

Formulations for rectal administration may be presented as a suppository with the usual carriers such as cocoa butter, synthetic glyceride esters or polyethylene glycol. Such carriers are typically solid at ordinary temperatures, but liquify and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

### EXAMPLES

### Preparation of EEV

EEV represents less than 1% of total virus produced in cell culture with many strains of VACV and is the only form of virus that is actively released from infected cells (Payne, 1980). The supernatant of infected cells is a source of EEV but this is often contaminated with IMV that has been released from infected cells. To generate EEV we used a cell culture system that produces high levels of extracellular virus but contains minimal contamination with IMV. Using this system, EEV from VACV strain WR-infected cells can be produced at 5×10⁷ plaque forming units (pfu) per ml with >70% of virus infectivity consisting of intact EEV (Law & Smith, 2004).

Confluent baby hamster kidney (BHK)-21 cells in a T175 flask were infected with VACV stain WR at 3 pfu/cell in Dulbecco's modified Eagle's medium (DMEM) supplemented with 10% heat inactivated foetal bovine serum (FBS) (50 IU/ml penicillin and 50 μg/ml streptomycin and 50 mM L-glutamine) for 2 h at 37° C., the virus inoculum was then washed away and the cells were incubated in 10 ml of DMEM with 2.5% FBS. At 24 h post-infection (pi) the culture supernatant was collected, centrifuged (10 min, 650×g) to remove detached cells and debris, and the supernatant was stored on ice. To determine the titre of infectious virus and the proportion of infectivity that is EEV, the virus was titrated by plaque assay on BS-C-1 cell monolayers before or after incubation with an IMV neutralizing mAb 2D5 as described previously (Law & Smith, 2001). For experiments that required a high titre of EEV (>10 pfu/cell), the extracellular virus present in the supernatant of infected cells was concentrated. The EEV outer membrane is fragile and many of the biophysical methods for concentrating or purifying EEV (such as velocity centrifugation in sucrose gradients) damage this outer membrane (Ichihashi, 1996; Vanderplasschen & Smith, 1997). EEV particles with a damaged outer membrane might behave as IMV, and therefore it is important to use methods that retain the EEV membrane integrity (Ichihashi, 1996; Vanderplasschen & Smith, 1997). EEV can be concentrated by centrifuging the infected cell supernatants in an ultracentrifuge (19,000×g, 80 min, 4° C.) and then very gently re-suspending the virions. By this method the virus is concentrated approximately 200-fold, the virions remain unaggregated, and the percentage of total virus that is EEV decreases by only 5% compared to that prior to centrifugation, indicating that the integrity of the EEV outer membrane is retained. This method has been used to concentrate EEV for binding studies and for examination of EEV by electron microscopy.

### Example 1

### Effect of Polyanions on EEV in the Presence or Absence of Antibody to IMV

### a) Using Antibody in Human Serum

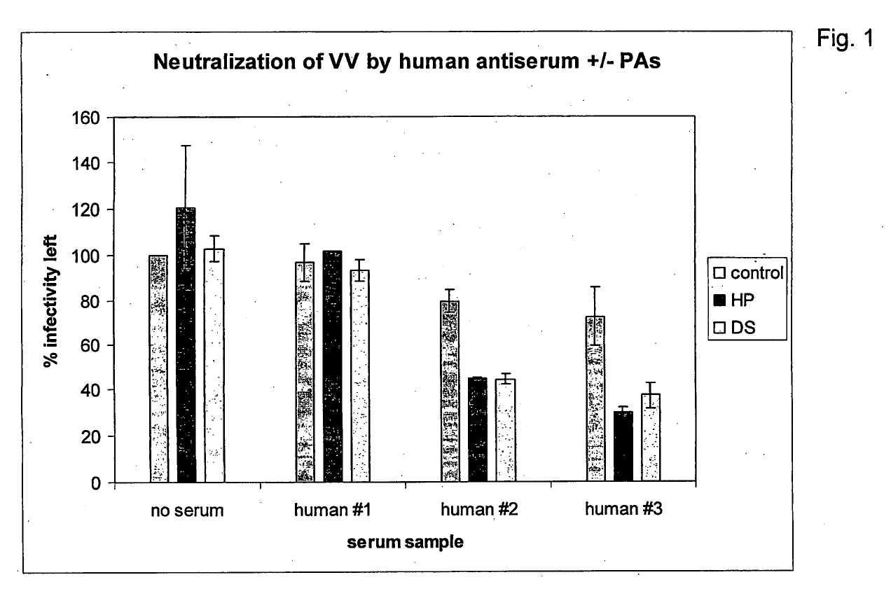

Using EEV prepared as described above, we tested human sera derived from either some-one who had not been immunized against smallpox and so was non-immune (serum #1), or from two humans who had been vaccinated against smallpox (sera #2 and #3). The 50% neutralization titres (ND₅₀) of these immune sera against IMV are 770 (serum 2) and 550 (serum 3). VACV harvested from supernatant of infected cells was incubated with human antiserum (diluted 1/100) with or without 25 μg/ml of heparin (HP, molecular weight (MW) 4,000-6,000) or dextran sulphate (DS, derived from dextran, (MW 5,000)). The results are shown in FIG. 1. Data shown are the mean of 2 experiments +/−S.D. In the presence of non-immune human serum, HP and DS did not reduce the infectivity of virus in the supernatant of VACV-infected cells, which represents predominantly EEV. In fact there was a slight increase in virus infectivity in the presence of these compounds. In the absence of PAs, the two immune sera reduced virus infectivity only moderately (from 21-27%), reflecting the inherent resistance of EEV to neutralization by antibody (Ichihashi, 1996; Vanderplasschen et al. 1997; Galmiche et al., 1999; Law & Smith, 2001). However, in the presence of PAs both antisera had greater neutralizing activity (55-61% for HP and 56-63% for DS), demonstrating synergistic activity of antibody and PAs.

### b) Using Monoclonal Antibody

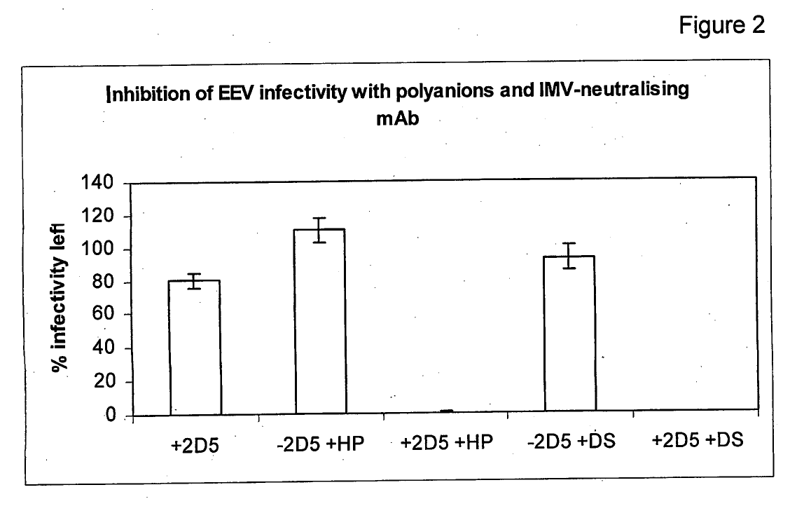

To examine this phenomenon further, and in particular to determine whether antibody to IMV or EEV was needed for the synergism with PAs, a similar assay was performed using mAb 2D5 that neutralizes IMV by interaction with the L1R protein (Ichihashi & Oie, 1996). In this laboratory mAb 2D5 is used routinely for the neutralisation of contaminating IMV from EEV preparations (Law & Smith, 2001). VACV was harvested from the supernatant of infected cells and was diluted to the required concentration to produce 200 pfu per cell monolayer of a 6-well plate. Virus was mixed at a 1:1 volume with 25 μg/ml HP (MW 4,000-6,000) or 25 μg/ml DS (derived from dextran, (MW 5,000)) or with no polyanion. IMV neutralising mAb 2D5 was diluted 1/1000 and was added to half of the samples. Samples were incubated for 1 h at 37° C. before the virus infectivity was determined by plaque assay on BS-C-1 cells. The results are shown in FIG. 2. Data shown are the mean of 2 experiments +/−S.D. The concentration of mAb 2D5 used inhibited >95% of purified IMV but only <30% of the infectivity in the supernatant of virus-infected cells. This indicated that the majority of virus in this preparation was EEV and the remainder represented contaminating IMV and/or EEV with a damaged membrane. Remarkably, in the presence of HP or DS, mAb 2D5 abolished virus infectivity in the supernatant, suggesting that PAs render EEV susceptible to the anti-IMV antibody.

### Example 2

### Effects of Different Polyanions

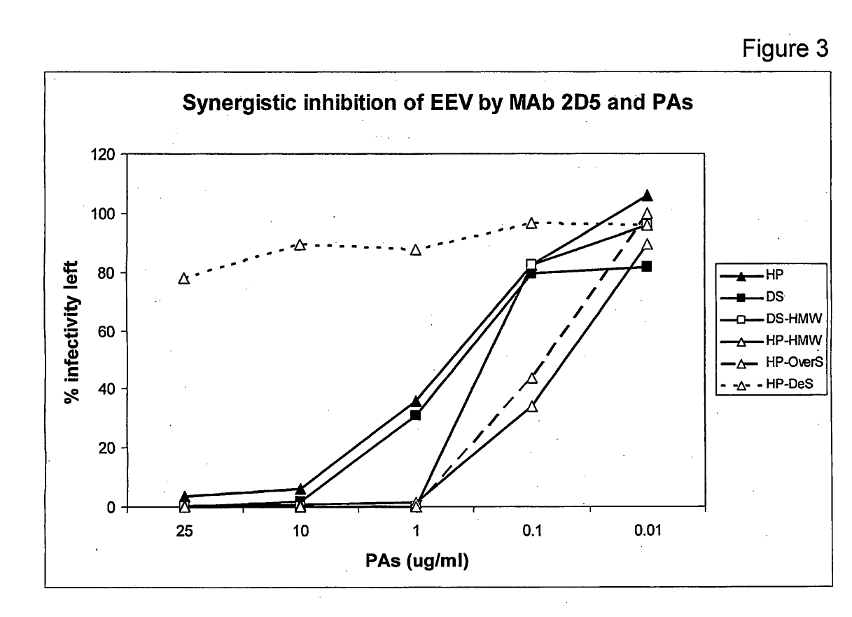

To investigate which types of PAs were most effective at rendering EEV sensitive to anti-IMV antibody a variety of compounds were tested using the method described above in Example 1(b). The infectivity of EEV treated with mAb 2D5 (diluted 1/1000) in the presence of HP (MW 4,000-6,000), DS. (derived from dextran 5,000), DS-HMW (MW 500,000), HP-HWM (MW 15,000), HP-OverS (over-sulphated HP-HWM) or HP-DeS (fully de-sulphated HP-HMW) was measured and the results are shown in FIG. 3. Data shown are the mean of two experiments. It was found that the synergistic effect of PAs was size and charge-dependent. High MW heparin (HP-HWM) (MW 15,000) and DS-HMW (MW 500,000) were more potent than HP and DS. Similarly, both HP and over-sulphated HP (HP-OverS) were more effective at the dose tested than desulphated HP (HP-DeS). The synergism of HP or DS and mAb 2D5 against EEV was potent: this is illustrated by the fact that a concentration of PAs of <1 μg/ml neutralized 50% of virus infectivity in the presence of mAb 2D5; In contrast, DS per se was reported to only partially inhibit VACV (most likely IMV form) at 80 μg/ml (Witvrouw et al., 1994).

### Example 3

### Effects of PAs and Anti-IMV Antibody on Comet Formation

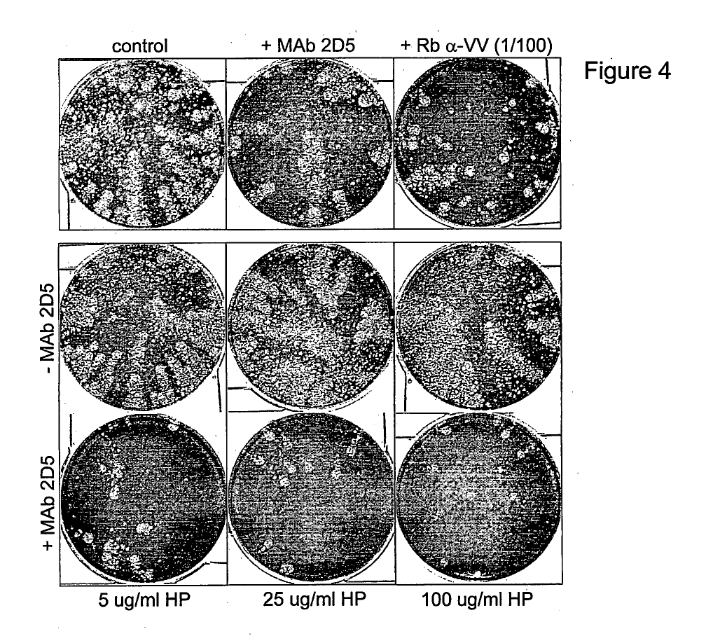

The ability of PAs and anti-IMV antibody to target the EEV envelope was also measured by an anti-comet assay. Comet-shaped plaques are formed when a VACV-infected cell monolayer is incubated in liquid overlay. Under these conditions, there is unidirectional dissemination of EEV from the primary infection site to form a series of secondary plaques by convection currents (Law et al., 2002). Agents that can prevent EEV spread will reduce the formation of comet-shaped plaques and this assay had been used for the measurement of anti-EEV activity of antibodies (Boulter & Appleyard, 1973). Monolayers of BSC-1 cells were infected with 25 pfu of VACV for 2 h. The cells were washed and overlayed with medium containing rabbit anti-VV antiserum (1/100), no antibody (shown in the —MAb 2D5 experiments) or mAb 2D5 (1/500), with or without HP (MW 4000-6000). HP was used at 5 μg/ml, 25 μg/ml or 100 μg/ml. Three days later the monolayers were stained with crystal violet solution. The results are shown in FIG. 4. When added alone, various concentrations of iP did not inhibit comet-shaped plaque formation, however, when mAb 2D5 was added, the comet tails were reduced severely and were almost abolished at the higher HP concentrations. The fact that comet formation was not inhibited by HP, but HP treatment is found to destroy the EEV membrane (see below), indicates that comet formation in the presence of HP is most likely mediated by IMV particles.

### Example 4

### Study of the Effects of Polyanions on Virus Binding and Entry

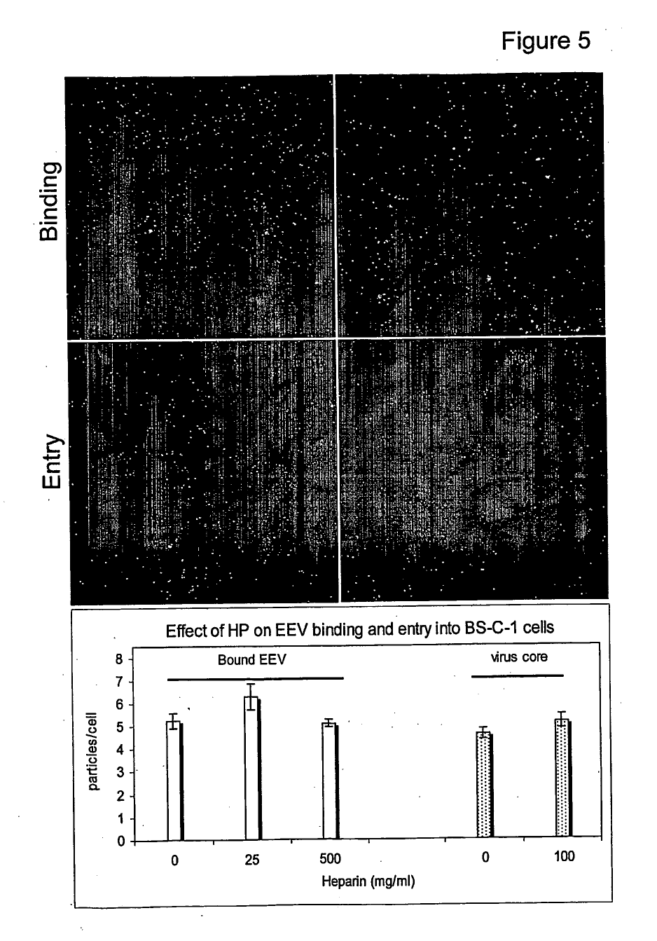

To investigate the mechanism of action of PAs, we tested if PAs affected the binding and entry of VACV. Fresh EEV particles were incubated with HP before binding to BS-C-1 cells. EEV in the presence or absence of HP was bound to BS-C-1 cells for 2 h on ice. Unbound virus was removed and cells were fixed to measure bound virions. EEV and virus cores were labelled with mAb 15B6 (specific to F13L protein) and rabbit anti-VV core antibody, respectively. The results are shown in FIG. 5 in which the upper panels show projected Z-series of confocal images of labelled EEV or virus cores in the presence or absence of HP (MW 4-6000, 50 μg/ml). Bar =50 μm.

To measure virus cores that had penetrated into the cytosol, cells were incubated with virus as above, washed and then incubated for 1 h at 37° C. to allow virus entry. Unbound virus was removed and cells were fixed to measure bound virions. Intracellular cores were labeled with rabbit anti-VACV core-specific antibody (Vanderplasschen et al., 1998a).

The bar chart in FIG. 5 shows the mean of bound EEV or virus cores +/−HP of six random microscopic views. Error bar =standard error. Surface virus particles and intracellular cores were quantified by confocal microscopy as described previously (Law & Smith, 2001; Carter et al., 2003; Law & Smith, 2004). The results showed that neither binding nor entry of virus was affected significantly by HP, indicating that the blockage is at a pre-binding stage.

### Example 5

### The Effect of Polyanions on Virus Replication

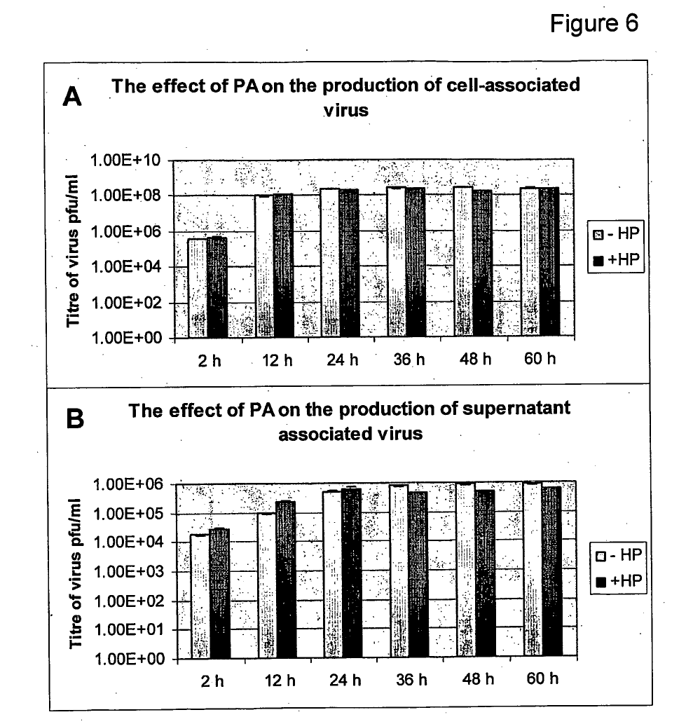

We investigated the effect of PAs on the formation and release of virus from infected cells. Twelve flasks of RK13 cells were infected at 10 pfu/cell with VACV strain WR for 2 h at 37° C. The virus inoculum was removed and the cells in half of the flasks were overlaid with medium containing 50 μg/ml HP. At various time points, 2, 12, 24, 36, 48 and 60 h, infected cells and supernatants from cells incubated with and without HP were collected. To determine the amount of virus present in the cell lysate (A) and the supernatants (B), samples were titrated on BS-C-1 cells as described above under “Preparation of EEV”. The results are shown in FIG. 6. Data shown are the mean of duplicate wells +/−S.D. In a time course experiment using RK₁₃ cells infected at 10 pfu/cell, no significant difference was observed in either the titre of virus that remained cell-associated (FIG. 6A) or was released into the culture medium (FIG. 6B) in the presence or absence of HP. The kinetics with which infectious virus was produced was also unaltered. This indicates that PAs did not affect the ability of the cells to produce infectious virions nor the infectivity of virus produced.

### Example 6

### PAs Reduce the Formation of Actin Tails at the Cell Surface

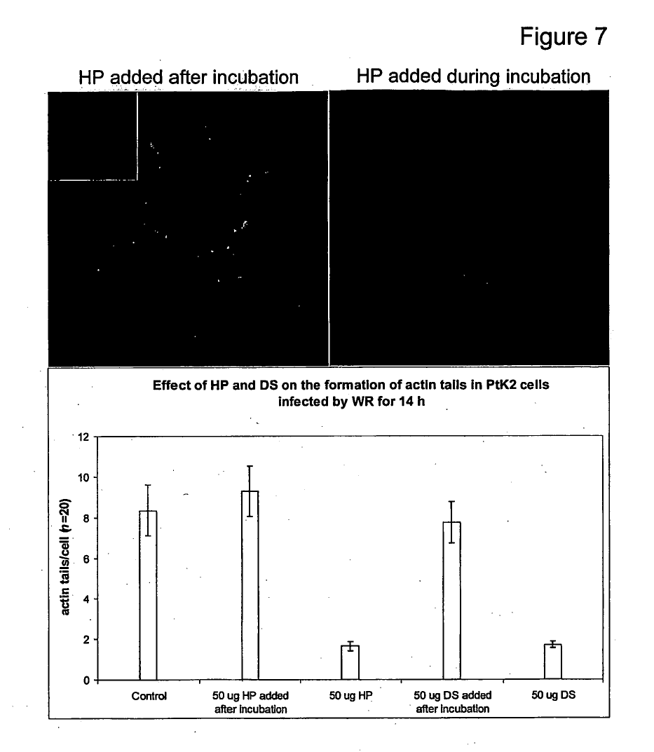

During VACV infection, IEV particles are transported to the cell surface on microtubules and after fusion of the plasma membrane with the IEV outer membrane, a CEV particle is exposed on the cell surface. CEV induce the formation of actin tails from beneath the plasma membrane where a CEV is attached, and these growing actin tails propel the enveloped virion away from the infected cell and into surrounding cells (Smith et al., 2003). Actin tails are important for efficient cell-to-cell spread of the virus and virus mutants that are defective in inducing actin tails produce only small plaques in vitro and are attenuated in vivo (Smith et al., 2002). To investigate if PAs affected actin tail formation from the surface of infected cells, HP or DS was added to cells infected with VACV during or after the course of infection. PtK2 cells were infected with VACV strain WR at 5 pfu/cell for 14 h. HP or DS (50 μg/ml) were added to the medium during or after the incubation. Actin and B5R were labelled with TRITC-phalloidin or rat mAb 19C2 and Cy5-conjugated donkey anti-rat IgG, respectively. Bar =10 μm. The results are shown in FIG. 7. The bar chart shows the levels of actin tails found in cells that made actin tails under the different conditions (Error bar =standard error). In the presence of HP and DS during incubation, fewer cells (reduced from ˜55% to 15%, n=100) were found to actually make actin tails and in cells that made actin tails significantly fewer actin tails were formed.

In summary of the results of Examples 4 to 6, PAs inhibit EEV infectivity in the presence of anti-IMV antibody before virus adsorption and inhibit virus dissemination by blocking actin tail formation. Induction of actin tails requires the retention of CEV on the cell surface to provide the signals for actin polymerization beneath the plasma membrane, whereas protection of IMV from IMV-neutralizing antibody requires the integrity of EEV membrane. Collectively, these data suggested that PAs damaged the EEV membrane.

### Example 7

### The Effects of PAs on the Integrity of the EEV Membrane

To investigate directly if PAs destroy the EEV membrane, EEV particles were incubated with HP (4-6000 MW), recovered by centrifugation and analysed by electron microscopy (EM). Supernatants were collected from 20 T175 flasks of BHK-21 cells that had been infected with VACV strain WR, 200 μg/ml of HP was added to half of the supernatants (100 ml) and the mixture was incubated at 37° C. for 1 h. The EEV particles present in each sample were collected by ultracentrifugation (19,000×g, 80 min, 4° C.) and resuspended gently in 1 ml of medium. These virions were recentrifuged in an eppendorf tube and the EEV pellets were fixed and processed for conventional Epon sectioning and transmission electron microscopy.

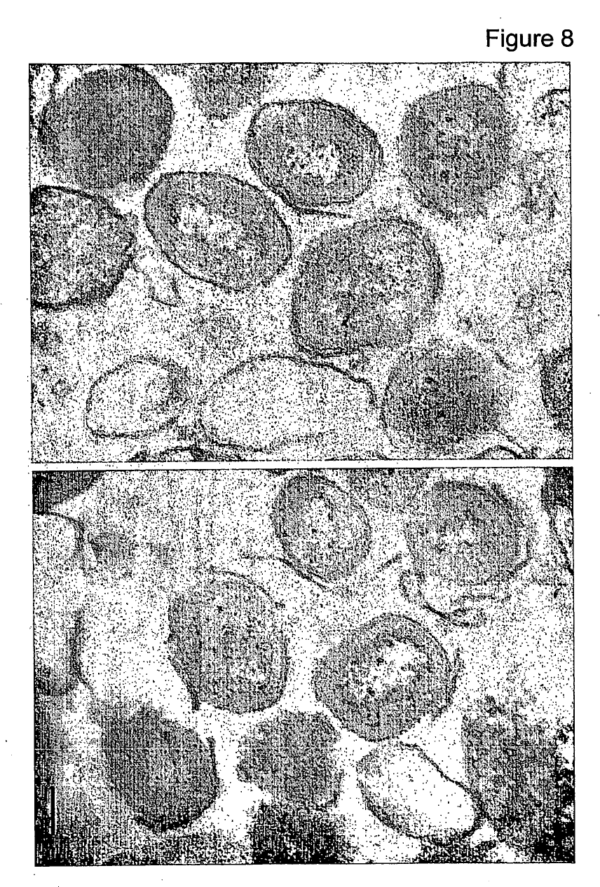

The samples were incubated with fixative (0.5% glutaraldehyde diluted in 200 mM sodium cacodylate) to preserve the structural detail and inactivate the live virus. The samples were centrifuged to form a pellet and processed into resin blocks from which 70 nm sections were cut and examined in an electron microscope. The results are shown in FIG. 8 in which A is an electron micrograph of EEV incubated without HP and B is an electron micrograph of EEV incubated with HP.

The ultracentrifugation procedure was found to damage less than 5% of the total EEV particles. In comparison to EEV treated in parallel without HP (FIG. 8A), it is clear that the outmost EEV membrane was severely damaged and the IMV inside was exposed (FIG. 8B) in EEV incubated with HP. The integrity of the EEV membrane was quantified by measuring the proportion of the circumference of virions that was covered by an EEV membrane. EEV particles that had not been treated with HP had 98.5% of their surface covered by EEV membrane (n=62 particles, standard deviation =5.68, standard error of the mean =0.72). In contrast, after treatment with heparin less than half (46.4%) of the surface of virions was associated with EEV membrane and these were only membrane fragments so that each virion had the IMV surface exposed in some places (n=46, standard deviation =23.3, standard error of the mean =3.44). Statistical analysis showed a significant difference (P<0.0001). The same result was obtained in a repeat experiment. These data demonstrate an unusual and novel function of PAs: namely, PAs damage the protective viral membrane and render the virus susceptible to antibody against internal components.

### Example 8

### The Effects of PAs on the CEV Outer Membrane on the Surface of Cells

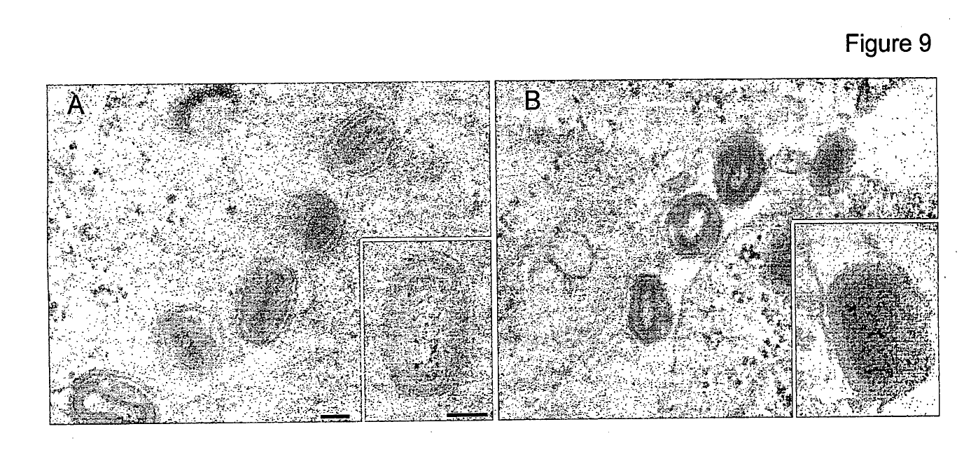

A further EM study was performed on infected cells. RK₁₃ cells were infected for 14 h as described in Example 5 in the absence or presence of HP (MW 4-6000) and the infected cells were prepared for electron microscopy as described previously (Hollinshead et al., 1999). The results are shown in FIG. 9. As seen in the figure, after infection with VACV strain WR, wrapped particles appear at the cell surface as CEV and these can become trapped between adjacent cells. The majority of these CEVs are identical to the isolated EEV particles having a tightly wrapped enveloping membrane, see inset (A). Cells that were infected at the same time but then incubated with 50 ug/ml HP also produced virions on the cell surface but these lacked an intact outer membrane and appeared as IMV. There was no evidence of cell lysis indicating that these had been not been released from lysed cells as IMV. Several of these particles could be seen to have severely damaged wrapping membranes see inset (B). The boundary of the cells had a different appearance due to the lack of actin tail formation on intact CEV particles (not seen). Scale bar =100 nm.

FIG. 9 shows that HP disrupted the outer membrane of CEV bound to the cell surface. We suggest that this might occur as soon as the virus was exposed on cell surface. In addition, actin tails were rarely seen in the presence of HP by EM, consistent with the confocal microscopic study.

### Example 9

### Effect of Pas on Poxvirus Infection In Vivo

Having demonstrated that PAs inhibit VACV by two novel mechanisms, namely (i) inhibition of actin tail formation, and (ii) rupture of the EEV membrane that protects the highly immunogenic IMV, we investigated whether PAs would confer benefit against poxvirus infection in vivo.

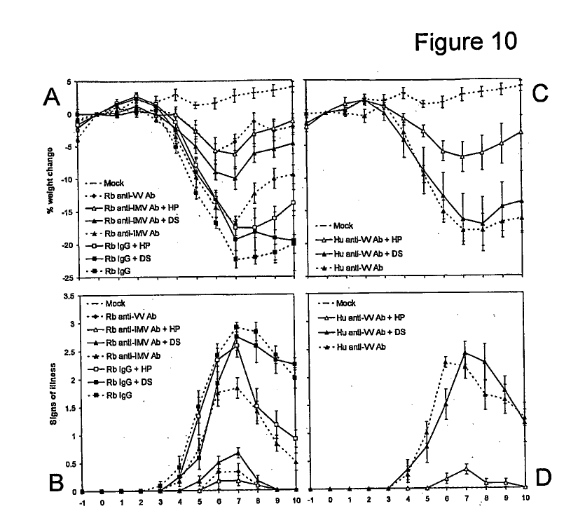

Groups of 6 mice were injected intraperitoneally with 200 μl PBS containing rabbit anti-VACV IgG (Rb anti-VV Ab, 500 μg), rabbit anti-VACV MV (Rb anti-IMV Ab, equivalent level of anti-IMV activity as Rb anti-VV Ab), rabbit non-immune IgG (Rb IgG, 500 μg) (A and B), or human anti-VACV IgG (Hu anti-VV Ab, 500 μg) (C and D), or PBS (Mock) (A-D), one day before infecting the mice intranasally with 1×10⁴ pfu of VACV strain WR. Two days post-infection, 20 μl of PBS, HP (2 mg) or DS (2 mg) were administered to the mice intranasally as indicated. The weights (A & C) and health status (signs of illness) (B & D) of the mice were recorded daily and the mice were monitored daily for weight change, signs of illness and virus replication in different organs as described previously (Williamson et al. 1990; Alcami & Smith, 1992; Tscharke et al., 2002; Reading & Smith, 2003). Data shown are the mean of 6 mice+/− standard error. The results are shown in FIG. 10 in which the solid lines represent groups that had been treated with PAs and the dashed lines represent groups that had not been treated with PAs.

PAs such as DS are known to be poorly absorbed in vivo, readily inactivated by serum, to have a short half-life, and induce toxic side-effects such as anticoagulation and causing thrombocytopoenia (Lüscher-Mattli, 2000). Despite these properties, administration of HP and DS did not cause weight loss or illness when administered up to 2 mg/mouse intranasally, whereas the same dose of high MW DS (MW 500,000) caused increased respiratory effort, followed by breathing difficulty and 15% body weight reduction after 2 days. Intraperitoneal injection of HP and high MW DS did not affect the mice adversely (unpublished data).

FIG. 10A shows that after infection animals given only non-immune IgG lost between 20 and 25% of their body weight before recovering. Those animals also given HP or DS alone lost slightly less weight loss. Animals immunized passively with anti-IMV IgG did slightly better than animals immunized with non-immune IgG, but this anti-IMV IgG conferred significantly less benefit than rabbit IgG raised against a live infection that would also contain antibody to EEV. This result was consistent with the reported greater importance of antibody to EEV (Boulter & Appleyard, 1973). Strikingly, in mice that had been immunised passively with a rabbit anti-IMV IgG (Rb anti-IMV Ab), HP and DS potentiated the therapeutic effect of this Ab, and HP and anti-IMV IgG provided protection to a level equivalent to rabbit IgG generated against live infection (Rb anti-VV Ab). The latter Ab contains anti-EEV antibody in addition to the IMV antibody and its concentration had been adjusted to give the same anti-IMV-neutralizing titre as the anti-IMV antibody. Therefore, these different IgGs differed only in the extra EEV antibody in the former. The severity of illness deduced by measurement of weight loss, was paralleled by that obtained by measuring the signs of illness (FIG. 10B) determined as described previously (Alcami & Smith, 1992). Next we investigated whether PAs conferred similar benefit in conjunction with human antibody (FIG. 10C & D). In the groups of animals that had received IgG from a human who had been vaccinated several times against smallpox, HP provided similar benefit, but DS was less effective. This antibody had 2.9 and 37-fold lower anti-IMV and anti-EEV titre than the rabbit hyperimmune antibody.

### Example 10

### The Effect of Pas and Anti-VACV Ab on the Titre of Infectious Virus in Infected Mice

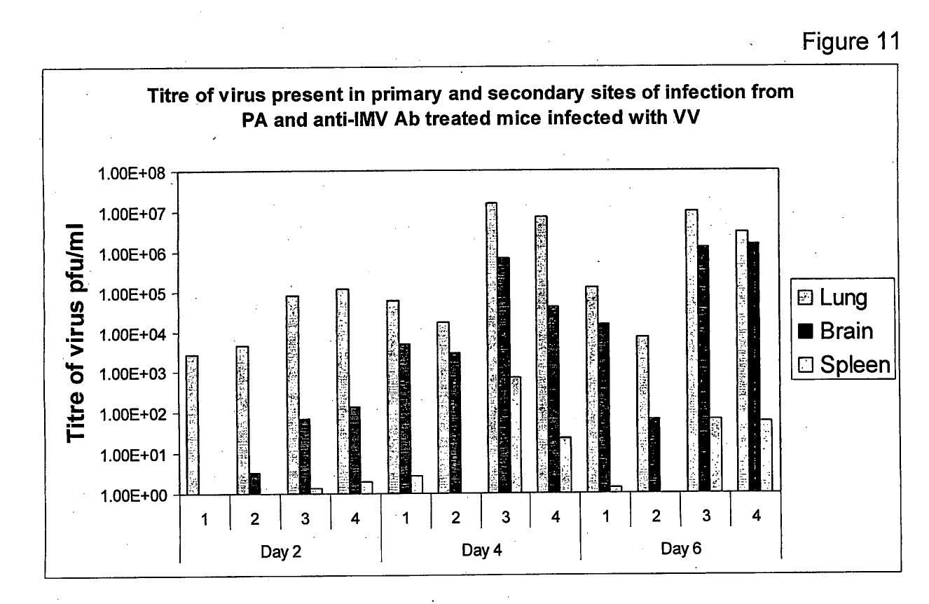

The severity of infection was also assessed by measurement of titres of infectious virus in primary (lungs) and secondary infection sites (spleen and brain). Groups of 9 mice were injected intraperitoneally with rabbit anti-VACV IMV Ab (groups 1 and 2) or rabbit non-immune IgG (groups 3 and 4), one day before infecting mice intranasally with 1×10⁴ pfu of VACV strain WR. Two days post infection, 20 μl of PBS (groups 1 and 3) or 2 mg HP (groups 2 and 4) were administered to mice intranasally. At 2, 4 and 6 days three mice were sacrificed, lungs, brains and spleens were removed and the amount of virus present in these organs was determined by plaque assay. The results are shown in FIG. 11. Data shown are the mean titres of virus (pfu/ml) produced from 3 mice.

We have shown that Rb anti-VACV Ab to be more effective than Rb anti-IMV Ab in limiting virus replication in lung and virus spreading to spleen but not to brain (M. Law & G. L. Smith unpublished data). In this study, Rb anti-IMV Ab alone restricted virus replication to a significant extent by day 2 (compare groups 1 and 2 with 3 and 4). On day 4 in the presence of HP but absence of anti-VACV Ab (group 4) the titres were lower than in the absence of HP, showing that HP alone had some benefit. But by day 6 it can be seen that animals treated with anti-VACV Ab and given HP (group 2) had lower titres of virus than groups given Ab alone (group 1). At this time, the levels of virus in the brain was reduced >100 fold and the level in lungs was reduced about 10-fold. Collectively, these data reinforced the observations on weight loss and signs of illness and demonstrate the benefit of PAs in treating poxvirus infections, especially in the presence of antibody to IMV surface proteins.

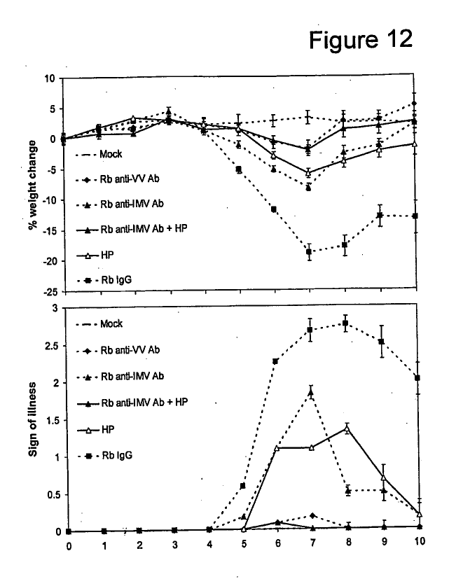

The benefit in vivo of HP and DS was confirmed in a second experiment that used a lower dose (3×10³ pfu) of virus challenge (FIG. 12). Groups of 6 mice were injected intraperitoneally with rabbit anti-VACV IgG (Rb anti-VV Ab), rabbit anti-VACV IMV (Rb anti-IMV Ab), rabbit non-immune IgG (Rb IgG) or PBS (Mock) one day before infecting the mice intranasally with 3×10³ pfu of VACV strain WR. On one and three days post-infection 20 μl of PBS or HP (2 mg) were administered to the mice intranasally as indicated. The weight and health status (signs of illness) of the mice were recorded daily. The results are shown in FIG. 12 in which the dashed lines represent groups that had been treated with PAs and the solid lines represent groups that had not been treated with PAs. Data shown are the mean of 6 mice+/− standard error.

These data validated the concept of treating a poxvirus infection with PAs and showed that PAs work synergistically with passively transferred anti-VACV-antibody.

### Example 11

### Investigation of the Requirement for B5R and A34R Proteins for Disruption of the EEV Membrane by PAs

A possible mechanism by which PAs affect the outer membrane of VACV CEV and EEV particles is illustrated in FIG. 13: In (A), EEV membrane protects the virus from IMV-neutralizing antibody but in the presence of PAs, interactions of PAs and EEV membrane proteins lead to the rupture of EEV membrane, rendering the virus susceptible to anti-IMV antibody. In (B) EEV membrane prevents the IMV within from interacting with cell for virus entry. Binding of EEV membrane proteins to cell surface ligands may lead to the rupture of EEV membrane by a mechanism similar to that of PAs. Once the EEV membrane is broken, the virus inside can enter cells as an IMV. This model predicts that there must be one of more molecules on the surface of the EEV and CEV particles with which polyanions interact. To address this, we utilized a collection of mutant viruses in which each of the genes encoding an EEV protein had been deleted individually (Blasco & Moss, 1991; Engelstad & Smith, 1993; McIntosh & Smith, 1996; Roper et al., 1998; Sanderson et al., 1998). Although these viruses produce different amounts of EEV compared to wild type and in some cases EEV production was reduced several fold, there was sufficient EEV made by these mutants for analysis.

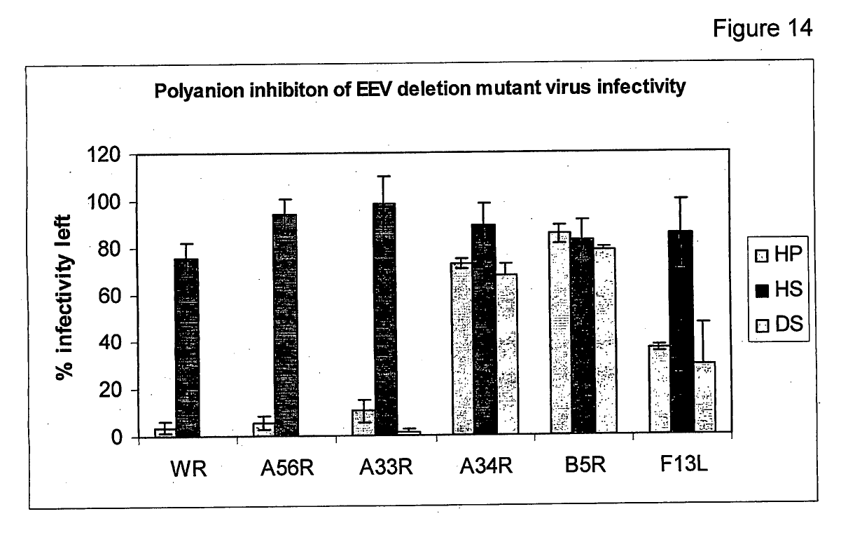

EEV from these mutant viruses was prepared as for wild type virus (as described in Preparation of EEV above) and the proportion of infectivity that was EEV was determined by plaque assay of untreated sample or after incubation in the presence of mAb 2D5. In parallel, samples were also treated with PAs (+/−mAb 2D5) and their infectivity was determined. BHK-21 cells were infected at 3 pfu/cell for 24 h with VACV stain WR or the mutant viruses ΔA56R, ΔA33R, ΔA34R, ΔB5R and ΔF13L. Virus was harvested from the supernatant of infected cells and was incubated with IMV neutralising mAb 2D5 (diluted 1/1000) and with or without 2 μg/ml of HP, HS or high molecular weight DS. Samples were incubated for 1 h at 37° C. before the virus infectivity was determined by plaque assay. The results are shown in FIG. 14. Data shown are the mean of 2 experiments +/−S.D.

It is seen in FIG. 14 that the deletion of the A33R and A56R genes did not alter the sensitivity of EEV to PAs and IMV-specific mAb compared to wild type. However, deletion of either the B5R or A34R gene and to a lesser extent the F13L gene made the EEV insensitive to PAs and IMV-neutralizing mAb. The F13L protein is present on the internal side of the EEV membrane and so it not located in a position to interact with PAs and therefore the effect of this protein on the sensitivity of EEV to PAs is likely to be indirect. However, the B5R and A34R proteins have the majority of their amino acids exposed on the outside of the EEV particle and so are available for interaction with PAs.

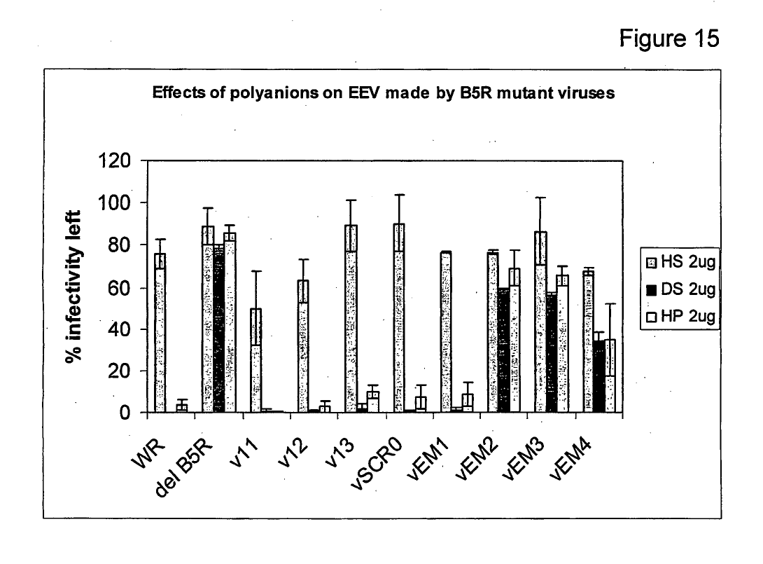

To investigate further which region of the B5R protein might be needed for an interaction with PAs, we utilized a series of mutant viruses in which the B5R protein had been altered. The B5R protein is an integral membrane glycoprotein that is embedded in the membrane with type I membrane topology; that is with the N-terminus exposed on the outside of the virion, a membrane-spanning sequence, and an internal C-terminus that is protected from the external medium by the EEV membrane (Isaacs et al., 1992; Engelstad & Smith, 1993). In the external domain the protein contains 4 short consensus repeat (SCR) domains that are typical of proteins that are members of a family of proteins that serve to regulate the activation of complement (Takahashi-Nishimaki et al. 1991). A series of deletion mutants with one or more of these domains removed (Herrera et al., 1998; Mathew et al., 1998) were analysed. Additional mutants in which either the short C-terminal region of the BSR protein was deleted, or in which the external, transmembrane or cytoplasmic domains of the protein had been swapped with the comparable domains of the A56R protein (Mathew et al., 2001) were analysed in parallel. BHK-21 cells were infected at 3 pfu/cell for 24 h with VACV strain WR and the mutant viruses with alterations in the B5R gene v11, v12, v13, vSCR0, vEM1, vEM2, vEM3 and vEM4. Virus was harvested from the supernatant of infected cells and was incubated with IMV neutralising mAb 2D5 (diluted 1/1000) and with or without 2 μg/ml of HP, HS or high molecular weight DS. Samples were incubated for 1 h at 37° C. before the virus infectivity was determined by plaque assay. The results are shown in FIG. 15. Data shown are the mean of 2 experiments +/−S.D.

It is seen in FIG. 15 that 5 of these mutants (v11, v12, v13, cSCR0 and vEM1) produced EEV that retained the sensitivity to PAs of wild type virus. In contrast, mutants vEM2 and vEM3 were resistant to PAs like vΔB5R, and one virus vEM3 had a phenotype intermediate between wild type and deletion mutant. Deletion mutants v11, v12, v13 and vSCR0 have lost 1, 2, 3 or all 4 SRC domains, respectively, but retain a short highly charged (acidic) domain close to the EEV membrane, the transmembrane sequence and the C-terminus (Herrera et al., 1998; Mathew et al., 1998). The phenotype of these mutants indicates that the only extracellular domain required for the EEV envelope to be sensitive to disruption by PAs is the charged (acidic) domain close to the EEV membrane. The phenotypes of the other mutants are broadly consistent with this.

One interpretation of these observations is that the B5R and A34R proteins are required for the PA-induced disruption of the EEV membrane and that the acidic region of the B5R protein close to the virus membrane is important for this. At neutral pH this region will be negatively charged. It is notable that the other protein, A34R, is predicted to have an opposite charge and so it is possible that these proteins have electrostatic interactions. An interaction between these proteins has been reported (Rottger et al., 1999). It is possible that highly charged polyanions may disrupt electrostatic interactions between the A34R and BSR proteins and that the lack of interaction between these proteins directly or indirectly mediates loss of membrane integrity.

The phenotype of the F13L protein is noteworthy in this regard. First, it has been reported to have phospholipase D activity and mutation of the protein to destroy this activity causes an interruption in wrapping of IMV to EEV (Husain & Moss, 2001). Second, the protein is present beneath the EEV membrane in EEV (Husain et al., 2003). It is conceivable that the protein may become activated by interaction of PAs with the A34R/B5R complex and that this results in disruption of the EEV membrane.

### REFERENCES

Alcami, A. & Smith, G. L. (1992). A soluble receptor for interleukin-1 beta encoded by vaccinia virus: a novel mechanism of virus modulation of the host response to infection. Cell 71, 153-167.
Anderson, S. G. & Skegg, J. (1970). The international standard for anti-smallpox serum. Bull World Health Organ 42, 515-523.
Armstrong, J. A., Metz, D. H. & Young, M. R. (1973). The mode of entry of vaccinia virus into L cells. Journal of General Virology 21, 533-537.
Blasco, R. & Moss, B. (1991). Extracellular vaccinia virus formation and cell-to-cell virus transmission are prevented by deletion of the gene encoding the 37,000-Dalton outer envelope protein. J Virol 65, 5910-5920.
Boulter, E. A. & Appleyard, G. (1973). Differences between extracellular and intracellular forms of poxvirus and their implications. Prog Med Virol 16, 86-108.
Bray, M. & Wright, M. E. (2003). Progressive vaccinia. Clin Infect Dis 36, 766-774.
Carter, G. C., Rodger, G., Murphy, B. J., Law, M., Krauss, O., Hollinshead, M. & Smith, G. L. (2003). Vaccinia virus cores are transported on microtubules. J Gen Virol 84, 2443-2458.
Chung, C. S., Hsiao, J. C., Chang, Y. S. & Chang, W. (1998). A27L protein mediates vaccinia virus interaction with cell surface heparan sulfate. J Virol 72, 1577-1585.
De Clercq, E. (2001). Vaccinia virus inhibitors as a paradigm for the chemotherapy of poxvirus infections. Clin Microbiol Rev 14, 382-397.
Earl, P. L., Americo, J. L., Wyatt, L. S., Eller, L. A., whitbeck, J. C., Cohen, G. H., Eisenberg, R. J., Hartmann, C. J., Jackson, D. L., Kulesh, D. A., Martinez, M. J., Miller, D. M., Mucker, E. M., Shamblin, J. D., Zwiers, S. H.,) Huggins, J. W., Jahrling, P. B. & Moss, B. (2004). Immunogenicity of a highly attenuated MVA smallpox vaccine and protection against monkeypox. Nature 428, 182-185.
Engelstad, M. & Smith, G. L. (1993). The vaccinia virus 42-kDa envelope protein is required for the envelopment and egress of extracellular virus and for virus virulence. Virology 194, 627-637.
Fenner, F., Wittek, R. & Dumbell, K. R. (1989). The Orthopoxviruses. London: Academic Press Ltd.
Fenner, F., Anderson, D. A., Arita, I., Jezek, Z. and Ladnyi, I. D. (1988). Smallpox and its eradication. Geneva: World Health Organization.
Galmiche, M. C., Goenaga, J., Wittek, R. & Rindisbacher, L. (1999). Neutralizing and protective antibodies directed against vaccinia virus envelope antigens. Virology 254, 71-80.
Gomez-Puertas, P. & Escribano, J. M. (1997). Blocking antibodies inhibit complete African swine fever virus neutralization. Virus Res 49, 115-122.
Gomez-Puertas, P., Oviedo, J. M., Rodriguez, F., Coll, J. & Escribano, J. M. (1997). Neutralization susceptibility of African swine fever virus is dependent on the phospholipid composition of viral particles. Virology 228, 180-189.
Gomez-Puertas, P., Rodriguez, F., Oviedo, J. M., Ramiro-Ibanez, F., Ruiz-Gonzalvo, F., Alonso, C. & Escribano, J. M. (1996). Neutralizing antibodies to different proteins of African swine fever virus inhibit both virus attachment and internalization. J Virol 70, 5689-5694.
Gordon, L. M., Waring, A. J., Curtain, C. C., Kirkpatrick, A., Leung, C., Faull, K. & Mobley, P. W. (1995). Antivirals that target the amino-terminal domain of HIV type 1 glycoprotein 41. AIDS Res Hum Retioviruses 11, 677-686.
Herrera, E., Lorenzo, M. M., Blasco, R. & Isaacs, S. N. (1998). Functional analysis of vaccinia virus B5R protein: essential role in virus envelopment is independent of a large portion of the extracellular domain. J Virol 72, 294-302.
Hollinshead, M., Vanderplasschen, A., Smith, G. L. & Vaux, D. J. (1999). Vaccinia virus intracellular mature virions contain only one lipid membrane. J Virol 73, 1503-1517.
Hooper, J. W., Custer, D. M., Schmaljohn, C. S. & Schmaljohn, A. L. (2000). DNA vaccination with vaccinia virus L1R and A33R genes protects mice against a lethal poxvirus challenge. Virology 266, 329-339.
Husain, M. & Moss, B. (2001). Vaccinia virus F13L protein with a conserved phospholipase catalytic motif induces colocalization of the B5R envelope glycoprotein in post-Golgi vesicles. J Virol 75, 7528-7542.
Husain, M., Weisberg, A. & Moss, B. (2003). Topology of epitope-tagged F13L protein, a major membrane component of extracellular vaccinia virions. Virology 308, 233-242.
Ichihashi, Y. (1996). Extracellular enveloped vaccinia virus escapes neutralization. Virology 217,478-485.
Ichibashi, Y. & Oie, M. (1996). Neutralizing epitope on penetration protein of vaccinia virus. Virology 220, 491-494.
Isaacs, S. N., Wolffe, E. J., Payne, L. G. & Moss, B. (1992). Characterization of a vaccinia virus-encoded 42-kilodalton class I membrane glycoprotein component of the extracellular virus envelope. J Virol 66, 7217-7224.
Krijnse-Locker, J., Kuehn, A., Schleich, S., Rutter, G., Hohenberg, H., Wepf, R. & Griffiths, G. (2000). Entry of the two infectious forms of vaccinia virus at the plasma membrane is signaling-dependent for the IMV but not the EEV. Mol Biol Cell 11, 2497-2511.
Law, M. & Smith, G. L. (2001). Antibody neutralization of the extracellular enveloped form of vaccinia virus. Virology 280, 132-142.
Law, M. & Smith, G. L. (2004). Studying the Binding and Entry of the Intracellular and Extracellular Enveloped Forms of Vaccinia Virus. In Methods Mol Biol, pp. 187-204. Edited by S. Isaacs.
Law, M., Hollinshead, R. & Smith, G. L. (2002). Antibody-sensitive and antibody-resistant cell-to-cell spread of vaccinia virus: role of the A33R protein in antibody-resistant spread. Journal of General Virology 83, 209-222.
Lüischer-Mattli, M. (2000). Polyanions—a lost chance in the fight against HIV and other virus diseases? Antivir Chem Chemother 11, 249-259.
Lüscher-Mattli, M., Gluck, R., Kempf, C. & Zanoni-Grassi, M. (1993). A comparative study of the effect of dextran sulfate on the fusion and the in vitro replication of influenza A and B, Semliki Forest, vesicular stomatitis, rabies, Sendai, and mumps virus. Arch Virol 130, 317-326.
Mathew, E., Sanderson, C. M., Hollinshead, M. & Smith, G. L. (1998). The extracellular domain of vaccinia virus protein B5R affects plaque phenotype, extracellular enveloped virus release, and intracellular actin tail formation. J Virol 72, 2429-2438.
Mathew, E. C., Sanderson, C. M., Hollinshead, R. & Smith, G. L. (2001). A mutational analysis of the vaccinia virus B5R protein. J Gen Virol 82, 1199-1213.
McIntosh, A. A. & Smith, G. L. (1996). Vaccinia virus glycoprotein A34R is required for infectivity of extracellular enveloped virus. J Virol 70, 272-281.
Mitsuya, H., Looney, D. J., Kuno, S., Ueno, R., Wong-Staal, F. & Broder, S. (1988). Dextran sulfate suppression of viruses in the HIV family: inhibition of virion binding to CD4+ cells. Science 240, 646-649.
Moss, B. (2001). Poxviridae: the viruses and their replication. In Virology, 4th edn, pp. 2849-2883. Edited by B. N. Fields, D. M. Knipe, P. M. Howley, R. M. Chanock, J. Melnick, T. P. Monath, B. Roizman & S. E. Straus. Philadelphia: Lippincott-Raven Publishers.
Payne, L. G. (1980). Significance of extracellular enveloped virus in the in vitro and in vivo dissemination of vaccinia. J Gen Virol 50, 89-100.
Reading, P. C. & Smith, G. L. (2003). A kinetic analysis of immune mediators in the lungs of mice infected with vaccinia virus and comparison with intradermal infection. J Gen Virol 84, 1973-1983.
Rodriguez, J. F., Janeczko, R. & Esteban, M. (1985). Isolation and characterization of neutralizing monoclonal antibodies to vaccinia virus. J Virol 56, 482-488.
Roper, R. L., Wolffe, E. J., Weisberg, A. & Moss, B. (1998). The envelope protein encoded by the A33R gene is required for formation of actin-containing microvilli and efficient cell-to-cell spread of vaccinia virus. J Virol 72, 4192-4204.
Rottger, S., Frischknecht, F., Reckmann, I., Smith, G. L. & Way, M. (1999). Interactions between vaccinia virus IEV membrane proteins and their roles in IEV assembly and actin tail formation. J Virol 73, 2863-2875.
Sanderson, C. M., Frischknecht, F., Way, M., Hollinshead, M. & Smith, G. L. (1998). Roles of vaccinia virus EEV-specific proteins in intracellular actin tail formation and low pH-induced cell-cell fusion. J Gen Virol 79 (Pt 6), 1415-1425.
Schmaljohn, C., Cui, Y., Kerby, S., Pennock, D. & Spik, K. (1999). Production and characterization of human monoclonal antibody Fab fragments to vaccinia virus from a phage-display combinatorial library. Virology 258, 189-200.
Smith, G. L., Vanderplasschen, A. & Law, M. (2002). The formation and function of extracellular enveloped vaccinia virus. J Gen Virol 83, 2915-2931.
Smith, G. L., Murphy, B. J. & Law, M. (2003). Vaccinia virus motility. Annu Rev Microbiol 57, 323-342.
Sodeik, B. & Krijnse-Locker, J. (2002). Assembly of vaccinia virus revisited: de novo membrane synthesis or acquisition from the host? Trends in Microbiology 10, 15-24.
Takahashi-Nishimaki, F., Funahashi, S., Miki, K., Hashizume, S. & Sugimoto, M. (1991). Regulation of plaque size and host range by a vaccinia virus gene related to complement system proteins. Virology 181, 158-164.
Tikunova, N. V., Morozova, V. V., Batanova, T. A., Belanov, E. F., Bormotov, N. I. & Ilyichev, A. A. (2001). Phage antibodies from combinatorial library neutralize vaccinia virus. Hum Antibodies 10, 95-99.
Tscharke, D., C., Reading, P. C. & Smith, G. L. (2002). Dermal infection with vaccinia virus reveals roles for virus proteins not seen using other inoculation routes. J Gen Virol 83, 1977-1986.
Vanderplasschen, A. & Smith, G. L. (1997). A novel virus binding assay using confocal microscopy: demonstration that the intracellular and extracellular vaccinia virions bind to different cellular receptors. J Virol 71, 4032-4041.
Vanderplasschen, A., Hollinshead, M. & Smith, G. L. (1997). Antibodies against vaccinia virus do not neutralize extracellular enveloped virus but prevent virus release from infected cells and comet formation. J Gen Virol 78 (Pt 8), 2041-2048.
Vanderplasschen, A., Hollinshead, M. & Smith, G. L. (1998a). Intracellular and extracellular vaccinia virions enter cells by different mechanisms. J Gen Virol 79, 877-887.
Vanderplasschen, A., Mathew, E., Hollinshead, M., Sim, R. B. & Smith, G. L. (1998b). Extracellular enveloped vaccinia virus is resistant to complement because of incorporation of host complement control proteins into its envelope. Proc Natl Acad Sci USA 95, 7544-7549.
Williamson, J. D., Reith, R. W., Jeffrey, L. J., Arrand, J. R. & Mackeft, M. (1990). Biological characterization of recombinant vaccinia viruses in mice infected by the respiratory route. J Gen Virol 71 (Pt 11), 2761-2767.
Witvrouw, M., Desmyter, J. & De Clercq, E. (1994). Antiviral portrait series: 4. Polysulfates as inhibitors of HIV and other enveloped viruses. Antivir Chem Chemother 5, 345-359.
Zsak, L., Onisk, D. V., Afonso, C. L. & Rock, D. L. (1993). Virulent African swine fever virus isolates are neutralized by swine immune serum and by monoclonal antibodies recognizing a 72-kDa viral protein. Virology 196, 596-602.

1. A composition comprising for simultaneous, sequential or separate administration:
a) a polyanion; and
b) an antibody reactive against an antigen on the surface of an intracellular form of a virus, which virus has an extracellular form that is surrounded by one lipid membrane more than the intracellular form.

2. A composition as claimed in claim 1 in which the virus that has an extracellular form that is surrounded by one lipid membrane more than the intracellular form, is selected from the chordopoxviruses.

3. A composition as claimed in claim 2 in which the chordopoxvirus is an orthopoxvirus.

4. A composition as claimed in claim 3 in which the orthopoxvirus is Variola virus, monkeypox virus, cowpox virus, camelpox virus or Vaccinia virus (VACV).

5. A composition as claimed in claim 3 or claim 4 in which the extracellular form is extracellular enveloped virus (EEV) and the intracellular form is intracellular mature virus (IMV).

6. A composition as claimed in claim 1, in which the polyanion has an Mr of from 400 to 1,000,000.

7. A composition as claimed in claim 1, in which the polyanion comprises a sulphated polysaccharide or a derivative thereof.

8. A composition as claimed in claim 7 in which the sulphated polysaccharide is selected from the group consisting of dextran sulphate, cellulose sulphate, heparin or heparin sulphate, dermatan sulphate, chondroitin sulphate, pentosan sulphate, fucoidin, mannan sulphate, carrageenan, dextrin sulphate, curdlan sulphate and chitin sulphate, and their derivatives.

9. A composition as claimed in claim 1, in which the antibody is directed to an IMV surface protein.

10. A composition as claimed in claim 9 in which the antibody is reactive against a protein selected from the group consisting of A27L, L1R, D8L, A28L, A17L, and H3L.

11-13. (canceled)

14. A method of treating a subject infected with a virus, which virus has an extracellular form that is surrounded by one lipid membrane more than the intracellular form, comprising administering to a subject in need thereof an effective amount of a composition comprising:
a) a polyanion, and
b) an antibody reactive against an antigen on the surface of an intracellular form of a virus, which virus has an extracellular form that is surrounded by one lipid membrane more than the intracellular form.

15. A method as claimed in claim 14 in which the virus is a chordopoxvirus.

16. A kit comprising in separate compartments:
a) a polyanion; and
b) an antibody reactive against an antigen on the surface of an intracellular form of a virus, which virus has an extracellular form that is surrounded by one lipid membrane more than the intracellular form.

17. A kit as claimed in claim 16 in which the virus is as a chordopoxvirus, the polyanion is a sulphated polysaccharide or a derivative thereof and the antibody is directed to an IMV surface protein.

18. (canceled)

19. A composition comprising a polyanion for the treatment of a subject infected with a virus, which virus has an extracellular form and an intracellular form, the extracellular form being surrounded by one lipid membrane more than the intracellular form whereby the subject is a subject that possesses antibodies against an antigen on the surface of an intracellular form of the virus.

20. A method of treating a subject infected with a virus, which virus has an extracellular form and an intracellular form, the extracellular form being surrounded by one lipid membrane more than the intracellular form, whereby the subject is a subject that possesses antibodies against an antigen on the surface of an intracellular form of the virus, comprising the step of administering to the subject in need thereof a composition comprising a polyanion.

21. A method of treating a subject comprising the steps of:
a) administering to the subject:
(i) a vaccine against a virus, which virus has an extracellular form and an intracellular form, the extracellular form being surrounded by one lipid membrane more than the intracellular form; or
(ii) an antibody against a virus, which virus has an extracellular form and an intracellular form, the extracellular form being surrounded by one lipid membrane more than the intracellular form; and
b) administering to the subject a polyanion.

22. A method as claimed in claim in which the virus is as a chordopoxvirus, and the polyanion is a sulphated polysaccharide or derivative thereof, and the antibody is directed to an IMV surface protein.

23. A method of neutralizing in vitro the infectivity of a virus, which virus has an extracellular form that is surrounded by one lipid membrane more than the intracellular form comprising the step of combining a test sample with a composition as claimed in claim 1.

24-26. (canceled)

27. A method as claimed in claim 18, in which the virus is selected from the group consisting of Variola virus, monkeypox virus, cowpox virus camelpox virus, and VACV.

28. A method as claimed in claim 18, in which the sulphated polysaccharide is selected from the group consisting of dextran sulphate, cellulose sulphate, heparin or heparin sulphate, dermatan sulphate, chondroitin sulphate, pentosan sulphate, fucoidin, mannan sulphate, carrageenan, dextrin sulphate, curdlan sulphate and chitin sulphate, and their derivatives.

29. A method as claimed in claim 18, in which the antibody is reactive against a protein selected from the group consisting of A27L, L1R, D8L, A28L, A17L, and H3L.
